(19) United States
(12) Patent Application Publication
(10) Pub. No.: US 2024/0287567 A1
(43) Pub. Date: Aug. 29, 2024

(54) ENZYMATIC METHOD FOR PREPARING REBAUDIOSIDE J

(71) Applicant: PepsiCo, Inc., Purchase, NY (US)

(72) Inventors: Alex TAO, Jiangsu (CN); Guoqing LI, Jiangsu (CN); Wenxia WANG, Jiangsu (CN); Leilei ZHENG, Jiangsu (CN); Chunlei ZHU, Jiangsu (CN); Xiaoliang LIANG, Jiangsu (CN); Kuikiu CHAN, Jiangsu (CN)

(21) Appl. No.: 18/600,332

(22) Filed: Mar. 8, 2024

Related U.S. Application Data
(62) Division of application No. 17/805,626, filed on Jun. 6, 2022, now Pat. No. 11,952,604.
Application No. 17/805,626 is a continuation of application No. 16/343,340, filed on Apr. 18, 2019, now Pat. No. 11,359,222.

(51) Int. Cl. C12P 19/56; C12N 9/10

(52) U.S. Cl. CPC C12P 19/56; C12N 9/1051; C12Y 204/01017

(57) ABSTRACT

Provided is a method for preparing Rebaudioside J using an enzymatic method, comprising using rebaudioside A as a substrate, and making the substrate, in the presence of a glycosyl donor, react under the catalysis of a UDP-glycosyltransferase-containing recombinant cell and/or UDP-glycosyltransferase prepared therefrom to generate Rebaudioside J.

### REFERENCE TO SEQUENCE LISTING SUBMITTED ELECTRONICALLY

The content of the electronically submitted sequence listing (Name: 3711_9320002_Seqlisting_ST26; Size: 5,096 bytes; and Date of Creation: Apr. 10, 2024) is herein incorporated by reference in its entirety.

### TECHNICAL FIELD

The present invention relates to a method for preparing Rebaudioside J, and in particular, relates to a biological method for preparing Rebaudioside J.

### BACKGROUND

Sweetening agents are a class of food additives that have wide applications in the production of food, beverages, and candies. They may be added in the food production process, or alternatively may be used through appropriate dilution as a substitute for sucrose in household baking. Sweetening agents include natural sweetening agents, for example, sucrose, high fructose corn syrup, honey, etc., and artificial sweetening agents, for example, aspartame, saccharine, etc. Steviosides are a class of natural sweetening agents extracted from the plant Stevia rebaudiana, and are widely used in food products and beverages at present. The extract of Stevia rebaudiana contains a variety of steviosides comprising rebaudioside. Naturally extracted steviosides have great differences in ingredients across different batches, and need subsequent purification.

The content of Rebaudioside J found in the steviosides of the Stevia leaves does not exceed 0.5%; thus, it is extremely difficult to obtain a extract of Rebaudioside J with high purity using the conventional method. Therefore, there are limited the in-depth studies of Rebaudioside J, and the commercial application of Rebaudioside J is hindered.

### SUMMARY

The technical problem to be solved by the present invention is to overcome the defects in the prior art. The present invention achieves so by providing a method for preparing Rebaudioside J using an enzymatic method. With such a method, rebaudioside J product with high purity can be produced at a lower cost and with a shorter production cycle.

The following technical solution is employed by the present invention to solve the technical problem described above.

Provided is a method for preparing Rebaudioside J using an enzymatic method, wherein in the method, rebaudioside A is used as a substrate; and in the presence of a glycosyl donor, Rebaudioside J is produced by means of reaction under the catalysis of recombinant cells containing UDP-glycosyltransferase and/or UDP-glycosyltransferase prepared therefrom. UDP-glycosyltransferase (i.e., uridine diphosphoglycosyltransferase), also referred to as UGT, is already well-known.

Preferably, the glycosyl donor is a rhamnosyl donor.

More preferably, the rhamnosyl donor is a UDP-rhamnose.

Preferably, the UDP-glycosyltransferase is a UGT-B from Oryza sativa (rice).

Preferably, the amino acid sequence of UGT-B from Oryza sativa is at least 60% consistent with Sequence 2 as shown in the Sequence Listing.

More preferably, the amino acid sequence of UGT-B from Oryza sativa is at least 70% consistent with Sequence 2 as shown in the Sequence Listing.

Further, the amino acid sequence of UGT-B from Oryza sativa is at least 80% consistent with Sequence 2 as shown in the Sequence Listing.

Furthermore, the amino acid sequence of UGT-B from Oryza sativa is at least 90% consistent with Sequence 2 as shown in the Sequence Listing.

According to one example, the amino acid sequence of UGT-B from Oryza sativa is completely identical with Sequence 2 as shown in the Sequence Listing.

According to the present invention, the reaction is carried out in an aqueous system at a temperature of 4-50° C. and a pH of 5.0 to 9.0. Preferably, the reaction is carried out in an aqueous system at a temperature of 35-45° C. and a pH of 7.5 to 8.5. More Preferably, the reaction is carried out at a temperature of below 40° C. and a pH of below 8.0.

More Preferably, the reaction is carried out in a phosphate buffer solution.

More Preferably, the reaction system contains recombinant cells of UDP-glycosyltransferase and a cell-permeating agent, and the reaction is carried out in the presence of the cell-permeating agent.

Further, the cell-permeating agent is toluene, and the volume ratio concentration of toluene in the reaction system is 1-3%. Furthermore, the volume ratio concentration of toluene is 2%.

More preferably, all the raw materials used in the reaction are added into a reaction kettle to be uniformly mixed and then placed at a set temperature for reaction while stirring. After the reaction is completed, a Rebaudioside J product which can meet the requirements for use can be obtained through purification-processing. A specific purification method is through post-processing including resin isolation; and a Rebaudioside J product with a purity as high as 95% can be obtained.

Preferably, the recombinant cell is a microbial cell.

More preferably, the microorganism is Escherichia coli, Saccharomyces cerevisiae, or Pichia pastoris.

By means of the foregoing technical solution, the present invention has the following advantages in comparison with the prior art:

The method of preparing Rebaudioside J using the enzymatic method provided by the present invention has important application values. As the substrate Rebaudioside A can be obtained in large quantities through using the enzymatic method, the production of Rebaudioside J is no longer limited by the quantity of raw materials. The production cost is thus greatly reduced. It should also be considered that because of the low content of Stevioside in the plant, and there are many Steviosides with different structures, it is rather difficult to extract a product with high purity. When compared with the prior art for extracting Rebaudioside J from Stevia leaves, the present invention provides a product with a higher purity by adopting the enzymatic synthesis method, which will promote the research and application of novel Stevioside Rebaudioside J.

### DETAILED DESCRIPTION OF THE INVENTION

For the structural formulas of Rebaudioside A and Rebaudioside J, see Formulas I and II respectively.

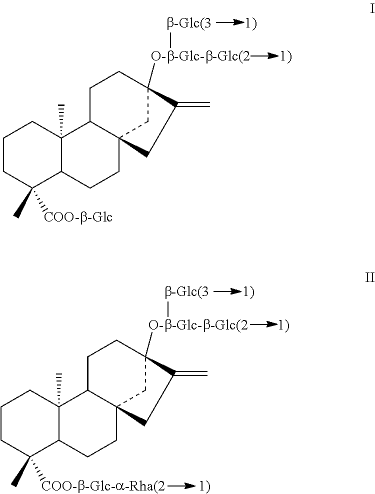

The main synthesis route of Rubadioside J as provided by the present invention is as follows:

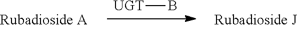

The UGT-B adopted in the present invention may exist in the form of lyophilized enzyme powder or in the recombinant cells.

The method for obtaining the UGT-B is as follows:
a recombinant Escherichia coli (or the other microorganisms) expression strain of UGT-B is obtained by utilizing molecular cloning technique and genetic engineering technique; then the recombinant Escherichia coli is fermented to obtain recombinant cells containing UGT-B, or to prepare and obtain lyophilized powder of UGT-B from the above noted recombinant cells.

Both the molecular cloning technique and the genetic engineering technique described in the present invention are already well-known. The molecular cloning technique may be found in Molecular Cloning: A Laboratory Manual (3rd Edition) (J. Sambrook, 2005).

The expression steps of the recombinant strain herein constructed by employing genetic engineering technique are as follows:
(1) (according to Sequence 1 and Sequence 2 as shown in the Sequence Listing) the required gene fragment is genetically synthesized, ligated into a pUC57 vector, while respectively adding Ndel and BamHI enzyme cutting sites at the two ends;
(2) each gene fragment is inserted into the corresponding enzyme cutting site of the expression vector pET30a through double digestion and ligation, so that each gene is placed under the control of T7 promoter;
(3) the recombinant plasmid is transformed into Escherichia coli BL21 (DE3); the expression of the target protein is induced by utilizing IPTG; and then the expression strains of the recombinant Escherichia coli of UGT-B is obtained.

The steps for preparing the recombinant cells containing UGT-B and the lyophilized powder of UGT-B by utilizing the expression strains of the recombinant Escherichia coli containing UGT-B are as follows:
the recombinant Escherichia coli expression strains containing UGT-B were inoculated into 4 ml of liquid LB medium according to a proportion of 1%; shake cultivation was carried out at 37° C. (at 200 rpm) overnight; the substance cultivated overnight was taken and inoculated into 50 ml of liquid medium according to a proportion of 1%; shake cultivation was carried out at 37° C. (at 200 rpm) overnight until the OD600 value reached 0.6-0.8; then IPTG with a final concentration of 0.4 mM was added in at 20ºC for shake cultivation overnight. After the induction is completed, the cells were collected through centrifugation (8,000 rpm, 10 min); then the cells were resuspended with 5 ml 2 mmol/L of phosphate buffer liquid (pH7.0), to obtain the recombinant cells; then the cells were ultrasonically disrupted in ice bath; the homogenate was centrifuged (8,000 rpm, 10 min); and the supernatant was collected and lyophilized for 24 h to obtain the lyophilized powder.

The present invention is further described in details in combination with specific examples.

### Example 1: Preparation of the Recombinant Escherichia Coli Cells Containing UGT-B

According to Sequence 1 and Sequence 2, the UGT-B gene fragment was genetically synthesized, while respectively adding Ndel and BamHI enzyme cutting sites at the two ends, and ligated to the pUC57 vector (produced by Suzhou Jin Wei Zhi Biotech. Co., Ltd.). The UGT gene segment was enzyme cut with restriction endonucleases Ndel and BamHI; and then the segments were recovered and purified; a T4 ligase was added to ligate the segments into the corresponding enzyme cutting sites of pET30a, in order to transform the BL21 (DE3) strains.

The UGT strains were inoculated into 4 ml of liquid LB medium according to a proportion of 1%; shake cultivation was carried out at 37° C. (at 200 rpm) overnight; the substance cultivated overnight was taken and inoculated into 50 ml of liquid LB medium according to a proportion of 1%; shake cultivation was carried out at 37° C. (at 200 rpm) overnight until the OD600 value reached 0.6-0.8; then IPTG with a final concentration of 0.4 mM was added in at 20° C. for shake cultivation overnight. After the induction is completed, the cells were collected through centrifugation (8,000 rpm, 10 min); and the collected cells were resuspended with 5 ml 2 mol/L of phosphate buffer (pH 7.0) to obtain the recombinant cells containing UGT-B for catalysis.

### Example 2: Preparation of the Lyophilized Powder of UGT-B

The recombinant cells containing UGT-B prepared in Example 1 were ultrasonically disrupted in ice bath; the homogenate was centrifuged (8,000 rpm, 10 min); and the supernatant was collected and lyophilized for 24 h to obtain the lyophilized powder of UGT-B.

### Example 3: Synthesis of Rebaudioside J Under the Catalysis of UDP-Glycosyltransferase with Rebaudioside a as the Substrate

In this example, lyophilized powder of UGT-B prepared according to the method of Example 2 was used for the catalysis and synthesis of Rebaudioside J.

1 L 0.05 mol/L of phosphate buffer solution (pH8.0), 2 g of UDP Rhamnose, 1 g of Rebaudioside A, 10 g of lyophilized powder of UGT-B were sequentially added into the reaction system, and placed into a water bath at 40° C. after evenly mixing, for reaction for 24 h while stirring at 300 rpm. After the reaction is completed, 500 μl of the reactant solution was added into anhydrous methanol of the equal volume for uniformly mixing; then it was centrifuged at 8,000 rpm for 10 min; and a high performance liquid chromatography was used to detect the supernatant filtration membrane (chromatographic conditions: column: Agilent eclipse sb-C18 4.6×150 mm; detection wavelength: 210 nm; mobile phase: acetonitrile: deionized water=24%: 76%; flow rate: 1.0 mL/min; column temperature: 30° C.). The conversion rate of Rebaudioside A was greater than 90%. After the supernatant was purified by post-processing such as isolating by silica resin and crystallizing, 0.52 g of Rebaudioside J was obtained, and the purity of which was greater than 95%.

### Example 4: Synthesis of Rebaudioside J Under the Catalysis of Recombinant Cells of UDP-Glycosyltransferase with Rebaudioside a as the Substrate

In this example, recombinant cells containing UGT-B prepared according to the method of Example 1 was used for the catalysis and synthesis of Rebaudioside J.

1 L 0.05 mol/L of phosphate buffer solution (pH8.0), 2 g of UDP Rhamnose, 1 g of Rebaudioside A, 20 ml of toluene, 40% of UGT-B whole cells were sequentially added into the reaction system, and placed into a water bath at 40° C. after uniformly mixing, to react for 24 h while stirring at 300 rpm. After the reaction is completed, 500 μl of the reactant solution was taken, and the supernatant was added with anhydrous methanol of the equal volume for uniformly mixing; then it was centrifuged at 8,000 rpm for 10 min; and a high performance liquid chromatography was used to detect the supernatant filtration membrane (chromatographic conditions: column: Agilent eclipse sb-C18 4.6×150 mm; detection wavelength: 210 nm; mobile phase: acetonitrile: deionized water=24%: 76%; flow rate: 1.0 mL/min; column temperature: 30° C.). The conversion rate of Rebaudioside A was greater than 90%. After the supernatant was purified by post-processing such as isolating by silica resin and crystallizing, 0.49 g of Rebaudioside J was obtained, and the purity of which was greater than 95%.

The above-described examples are merely for the illustration of the technical concept and features of the present invention. The object of providing examples is only to allow those skilled in the art to understand the present invention and implement it accordingly; the scope of the present invention is not limited thereto. Any equivalent variations or modifications derived from the essence of the present invention shall fall within the protection scope of the present invention.

What is claimed is:

1. A recombinant cell comprising a UDP-glycosyltransferase, wherein the UDP-glycosyltransferase comprises an amino acid sequence having at least 80% sequence identity to SEQ ID NO: 2.

2. The recombinant cell of claim 1, wherein the UDP-glycosyltransferase comprises an amino acid sequence having at least 90% sequence identity to SEQ ID NO: 2.

3. The recombinant cell of claim 2, wherein the recombinant cell is selected from the group consisting of Escherichia coli, Saccharomyces cerevisiae, and Pichia pastoris.

4. The recombinant cell of claim 2, wherein the UDP-glycosyltransferase is a uridine diphosphoglycosyltransferase-B (UGT-B) from Oryza sativa.

5. A method of preparing a lyophilized powder of UDP-glycosyltransferase, comprising disrupting the recombinant cell of claim 2, obtaining a cell homogenate, centrifuging the cell homogenate to obtain a supernatant, and lyophilizing the supernatant.

6. The method of claim 5, wherein the recombinant cell is selected from the group consisting of Escherichia coli, Saccharomyces cerevisiae, and Pichia pastoris.

7. The method of claim 5, wherein the UDP-glycosyltransferase is a uridine diphosphoglycosyltransferase-B (UGT-B) from Oryza sativa.


SEQUENCE LISTING (2 sequences)

SEQ ID NO: 1 - DNA, 1389 bp, synthetic construct
  source 1..1389: mol_type = other DNA; note = Synthesized nucleotide sequence
atggacagcggttactcttctagctatgctgcggcagccggtatgcacgtagttatttgtccgtggctcgctttcggtcacctcctgccgtgcctggacctggcgcagcgcctggcatctcgtggtcaccgtgtcagtttcgttagcacgccgcgtaacatctcacgtctgccgccggtccgtccggctctggccccgctggttgcgttcgttgcgctacctctgccgcgcgttgaaggcttaccggatggcgcagagtctaccaacgacgtgccgcacgatcgcccggatatggttgaactccaccgccgtgcatttgacggtctggcagctccgttctccgaatttctgggtaccgcgtgtgccgactgggtcatcgtagacgtattccaccactgggcagctgcagcggctttagaacacaaagtaccgtgcgcaatgatgctgctgggctctgctcacatgatcgcgtctattgccgaccgtcgtctggaacgtgcagagaccgaatctccagcggcagccggtcagggccgtcctgcagctgctccgaccttcgaagttgctcgtatgaagctcatccgcactaaaggttcttccggtatgtcactggcagagcgtttctcgctgacgctctcccgtagcagcctggttgtggggcgctcctgcgtggaattcgaaccggaaactgtgccgctactgtctaccctgcgtggcaagccgatcacttttctgggtctcatgccgccactgcacgaaggtcgccgcgaagacggtgaagatgctacggttcgttggttggacgcccagccggctaaaagcgtcgtgtacgtagctctgggcagtgaagttccattgggtgtcgagaaagtgcatgaactggctttgggtctggagctggctggcacccgtttcctctgggcactgcgtaagccgactggtgtgtctgatgctgaccttctgccggctggtttcgaagaacgtacccgtggtcgcggcgtagtggcaacccgctgggtaccgcagatgtccatcctggcacacgctgctgttggcgcgtttcttacccactgcgggtggaactctacaatcgaaggcctgatgttcggccatcctctgattatgctgccaatcttcggtgatcagggtccgaacgctcgtctgatcgaagccaaaaacgccggcttacaagtcgcacgcaacgacggcgatggttctttcgatcgtgaaggtgttgcggcagctatccgtgcagtggctgtagaagaagagtcgagcaaagtgttccaggcaaaagccaaaaagctgcaggaaatcgttgcggacatggcgtgccacgaacgttacatcgatggctttatccagcagctgcgctcctacaaagattaa

SEQ ID NO: 2 - protein, 462 aa, synthetic construct
  source 1..462: note = Synthesized protein sequence
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKD